(19) United States
(12) Patent Application Publication
(10) Pub. No.: US 2010/0055759 A1
(43) Pub. Date: Mar. 4, 2010

(54) TRANSPARENT POLYMER MEMBRANE FOR LASER DISSECTION

(76) Inventors: Helen M. Blau, Menlo Park, CA (US); Karen Havenstrite, Palo Alto, CA (US)

Correspondence Address: THOMAS, KAYDEN, HORSTEMEYER & RISLEY, LLP, 600 GALLERIA PARKWAY, S.E., STE 1500, ATLANTA, GA, 30339-5994, US

(21) Appl. No.: 12/552,418

(22) Filed: Sep. 2, 2009

Related U.S. Application Data
(60) Provisional application No. 61/094,248, filed on Sep. 4, 2008.

(51) Int. Cl. C12N 13/00; C12N 5/06

(52) U.S. Cl. 435/173.9; 435/378

(57) ABSTRACT

The disclosure encompasses transparent polymer membranes suitable for use in laser microdissection methods, where the membranes allow the subsequent analysis of the microdissected cell or cells with light of a wavelength that can traverse the membrane with little if any absorbance by the membrane. The transfer capture membranes of the disclosure comprise a polymeric composition having the characteristic of being cut by a laser light at a wavelength less than 360 nm, while being substantially optically transparent at a wavelength greater than about 360 nm. The disclosure further provides methods of isolating a cell or population of cells from a tissue, comprising: overlaying a tissue sample with a transfer capture membrane having the characteristic of being cut by a laser light at a wavelength less than 360 nm, and substantially optically transparent at a wavelength greater than about 360 nm, defining an area of the transfer capture membrane by a laser light cutting through the thickness of the transfer capture membrane and the issue sample; and removing the defined area of the transfer capture membrane with the targeted cell or population of cells adhering thereto.

### CROSS-REFERENCE TO RELATED APPLICATIONS

This application claims priority to U.S. Provisional Patent Application Ser. No.: 61/094,248, entitled “TRANSPARENT POLYMER MEMBRANE FOR LASER DISSECTION” filed on Sep. 4, 2008, the entirety of which is hereby incorporated by reference.

### TECHNICAL FIELD

The present disclosure is generally related to materials and methods of use thereof for laser dissection of biological tissues.

### BACKGROUND

To fully understand the etiology and pathogenesis of a disease, it would often be of benefit to isolate and characterize a specific population of cells from the complex mix of cell types of most tissues. Although biochemical and genetic differences between cells in a tissue may be identified by histological techniques, the isolation and extraction of proteins and nucleic acids from individual cells or small groups of related cells can be valuable in associating specific factors to localized tissue heterogeneities.

An especially useful technique for the selection and isolation of single cells or small groups of cells from a tissue is that of laser microdissection or laser capture microdissection. In this procedure, a section of a tissue is mounted on a slide and overlaid with a polymer film. The area of the section of interest is then identified, and a laser beam used to cut the film around the cell area to be extracted. The cells adhere to the film, which is then lifted away, bearing the adherent cell or cells.

Current membrane materials for use in laser capture microdissection include polyethylene terephthalate and polyethylene naphthalate (PEN) thin films. These membranes are extremely porous and the optical properties of the membranes are not well-suited for techniques that may be applied to cells adhering to the membrane, such as DAPI staining.

It is difficult to image cells on these membranes. The PEN material is designed to be efficient for laser microdissection because it absorbs energy in the range of the laser (i.e., about 355 nm). However, it also absorbs light at about 380 nm which prevents, for example, DAPI staining of the cell membranes. DAPI or 4′,6-diamidino-2-phenylindole is a fluorescent stain that binds strongly to DNA and is used extensively in fluorescence microscopy. Since DAPI will pass through an intact cell membrane, it may be used to stain both live and fixed cells.

For fluorescence microscopy, DAPI is excited with ultraviolet light. When bound to double-stranded DNA, its absorption maximum is at 358 nm and its emission maximum is at 461 nm. (This emission is fairly broad, and appears blue/cyan.) DAPI will also bind to RNA, though it is not as strongly fluorescent. Its emission shifts to around 500 nm when bound to RNA. These are both significant limitations of the current membranes.

### SUMMARY

Embodiments of the present disclosure encompass transparent polymer membranes suitable for use in laser microdissection methods, where the membranes allow the subsequent analysis of the microdissected cell or cells with light of a wavelength that can traverse the membrane with little if any absorbance by the membrane.

One aspect of the disclosure encompasses transfer capture membrane for laser microdissection of a biological tissue sample, wherein the membrane can comprise a polymeric composition having the characteristic of being cut by a laser light at a wavelength less than 360 nm, wherein the capture membrane is substantially optically transparent at a wavelength greater than about 360 nm.

In embodiments of the transfer capture membrane of the disclosure, the polymeric composition can comprise a photoreactive epoxy resin.

In one embodiment of the transfer capture membrane of this aspect of the disclosure, where the polymeric composition can comprise the photoreactive epoxy resin SU-8.

In embodiments of the transfer capture membrane of the disclosure, the membrane can have a thickness between about 1 micron and about 20 microns.

In some embodiments of the transfer capture membrane of the disclosure, the membrane can have a thickness between about 1 micron and about 10 microns.

In some embodiments of the transfer capture membrane of the disclosure, the membrane can have a thickness between about 4 microns and about 10 microns.

Another aspect of the disclosure encompasses methods of isolating a cell or population of cells from a tissue, comprising: overlaying a tissue sample with a transfer capture membrane, where the transfer capture membrane comprises a polymeric composition having the characteristic of being cut by a laser light at a wavelength less than 360 nm, and where the capture membrane is substantially optically transparent at a wavelength greater than about 360 nm, and where a cell or a population of cells adheres to the transfer capture membrane; defining an area of the transfer capture membrane with a laser light, where the laser light cuts through the thickness of the transfer capture membrane and the issue sample; and removing the defined area of the transfer capture membrane, thereby isolating the cell or population of cells from the tissue sample.

In embodiments of the methods of this aspect of the disclosure, the polymeric composition can comprise a photoreactive epoxy resin.

In one embodiment of the methods of this aspect of the disclosure, the polymeric composition can comprise the photoreactive epoxy resin SU-8.

In embodiments of the methods of this aspect of the disclosure, the membrane can have a thickness between about 1 micron and about 20 microns.

In embodiments of the methods of this aspect of the disclosure, the membrane can have a thickness between about 1 micron and about 10 microns.

In embodiments of the methods of this aspect of the disclosure, the membrane can have a thickness between about 4 microns and about 10 microns.

Still another aspect of the disclosure provides kits comprising materials to prepare a transfer capture membrane comprising a polymeric composition having the characteristic of being cut by a laser light at a wavelength less than 360 nm, where the capture membrane is substantially optically transparent at a wavelength greater than about 360 nm, packaging, and instructions for combining the materials to form a transfer capture membrane.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

It must be noted that, as used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

As used herein, the following terms have the meanings ascribed to them unless specified otherwise. In this disclosure, “comprises,” “comprising,” “containing” and “having” and the like can have the meaning ascribed to them in U.S. Patent law and can mean “includes,” “including,” and the like; “consisting essentially of” or “consists essentially” or the like, when applied to methods and compositions encompassed by the present disclosure refers to compositions like those disclosed herein, but which may contain additional structural groups, composition components or method steps (or analogs or derivatives thereof as discussed above). Such additional structural groups, composition components or method steps, etc., however, do not materially affect the basic and novel characteristic(s) of the compositions or methods, compared to those of the corresponding compositions or methods disclosed herein. “Consisting essentially of” or “consists essentially” or the like, when applied to methods and compositions encompassed by the present disclosure have the meaning ascribed in U.S. Patent law and the term is open-ended, allowing for the presence of more than that which is recited so long as basic or novel characteristics of that which is recited is not changed by the presence of more than that which is recited, but excludes prior art embodiments.

In describing and claiming the disclosed subject matter, the following terminology will be used in accordance with the definitions set forth below.

### Discussion

Embodiments of the present disclosure encompass transparent polymer membranes suitable for use in laser microdissection methods, where the membranes allow the subsequent analysis of the microdissected cell or cells with light of a wavelength that can traverse the membrane with little if any absorbance by the membrane.

Laser microdissection is an extraction process to dissect specific tissue, cells or poplations of cells from a thin tissue sample for analysis. In this procedure, a laser is coupled into a microscope and focuses onto the tissue of the slide. By movement of the laser by optics or the stage of the microscope, the focus follows a trajectory which is predefined by the user. This area of the tissue defined by the laser ablation, is then cut out and separated from the adjacent tissue. There are several ways to extract tissue from a microscope slide with a histopathology sample on it: In particular, and suitable for the methods of the present disclosure, a sticky surface may be pressed onto the sample and then lifted. This will extract the desired region, but also bears the chance to carry particles or unwanted tissue. Alternatively, a plastic membrane may be melted onto the sample and then removed. The heat is introduced by an, e.g., red or IR laser onto a membrane stained with an absorbing dye. As this adheres the desired sample onto the membrane, as with any membrane that is put close to the histopathology sample surface, there might be some debris extracted. Another danger is the introduced heat damaging the sensitive biological material. Techniques for laser microdissection for which the membranes of the disclosure are useful are discussed in, for example, Espina et al., (2006) Nature Protocols 1: 586-603, which is incorporated herein by reference in its entirety.

While the membranes of the disclosure absorb light of wavelengths below 360 nm, thereby allowing laser cutting of the membrane, they are also substantially transparent to light above about 365 nm wavelength, a property that allows their use as a supporting substratum of microdissected cells in observational orimaging techniques requiring the higher wavelength values. In addition, embodiments of the present disclosure encompass methods of laser dissection using embodiments of the transparent polymer membranes of the present disclosure. Further, embodiments of the present disclosure include use of transparent polymer membranes that can be used in imaging applications where a thin membrane that is optically clear is required.

Embodiments of the laser microdissection membranes of the disclosure comprise a photoactive epoxy resin (such as, but not limited to, SU-8) that can be fabricated into thin uniform sheets. Epoxy resins suitable for use in the preparation of the films of the present disclosure include, but are not limited to, multifunctional epoxy resins and multifunctional acrylate resins. The multifunctional epoxy resin may include, but is not limited to, any resin having two or more functional groups and containing an oxirane group. Specific examples thereof may include, but may be not limited to, bisphenol A type epoxy resins, bisphenol F type epoxy resins, hydroquinone type epoxy resins, resorcinol type epoxy resins, and novolac type epoxy resins, which may be used alone or in mixtures of two or more thereof. Commercially available examples of the epoxy resin may include EPON 828, EPON 1004, EPON 1001 F, EPON 1010, and EPON SU-8, available from Shell Chemicals, DER-331, DER-332, DER-334, DEN-431, and DEN-439, available from Dow Chemical Company, and ERL-4201, ERL-4289, and ERL-0400, available from Union Carbide Corporation.

SU-8 was originally developed for the microelectronics industry to provide a high resolution negative imaging resist for use in the fabrication of advanced semiconductor devices. The photosensitivity of SU-8 is 300-400 nm. Because of the highly cross-linked matrix in the exposed material, it is thermally stable (up to 200° C.) and chemically stable after development, and is soluble in a variety of organic solvents.

For example, SU-8 is composed of three components; an EPON epoxy resin, an organic solvent, and a photoinitiator. The chemical formula of EPON resin SU-8 is a multifunctional glycidyl ether derivative of bisphenol-A novolac used to provide high-resolution patterning for semiconductor devices. The second one is gamma-butyrolactone (GBL), an organic solvent. The quantity of the solvent determines the viscosity of the solvent, which determines final thickness of the spin-coated film. Along with GBL, cyclopentanone is also used as a solvent for later products of SU-8 (SU-8 2000 series). And the third one is triarylium-sulfonium salts (CYRACURE® UVI from Union Carbide), a photoinitiator which is approximately 10 wt % of EPON SU-8. Epoxy resins could be cationically polymerized by utilizing a photoinitiator which generates strong acid upon exposure to ultraviolet light (365 to 436 nm) and the acid facilitates polymeric cross-linking during post-exposure bake.

The starting material is supplied as a liquid consisting of an epoxy resin, a solvent (GBL or cyclopentanone depending on formulation) and a photo-acid generator. A fil may be formed using a conventional photoresist spinner, with the film thickness controlled by the spin speed and the solids content of the epoxy solution. A baking stage can removes excess solvent from the layer. Upon exposure to UV radiation, a strong acid (HSbF₆) is generated which causes the epoxy resin to form a ladder-like structure with a high cross-linking density when heated above a critical temperature provided in a post-exposure bake. The material unexposed to a UV light source is then removed with a solvent in the development process.

The amount of the multifunctional epoxy resin may be determined according to an appropriate choice made by one skilled in the art depending on the end use and need, and may be set in the range of about 15 to about 90 wt %, for example, about 30 to about 75 wt %, based on the total amount of the photosensitive composition. As such, an amount of the epoxy resin of about 15 wt % or more may enable the formation of a more rigid film.

Embodiments of the photoactive epoxy resin can be used as a lift-off for laser microdissection applications. In the embodiments of the disclosure, it is understood that the photoactive epoxy resin includes photoactive epoxy resins that are substantially transparent to light above about 365 nm wavelength, but absorb light below about 360 nm. In an embodiment, the photoactive epoxy resin includes photoactive epoxy resins that have an absorbance peak at about 360 nm.

The thin films herein disclosed are less than about 5 microns, less than about 4 microns, less than about 3 microns, or less than about 2 microns in thickness. In one embodiment, the film or material used to form the film (that is, a photoactive epoxy resin) has the characteristic of absorbing laser energy at 355 nm but is transparent to light having wavelengths above about 365 nm, thereby enabling both high efficiency laser cutting and the ability to visualize fluorescent staining at wavelengths greater than 365 nm.

The film or material used to form the film (photoactive epoxy resin) is optically clear to enable microscopic visualization of cells. Embodiments of the present disclosure can be used for microdissection as a liftoff mechanism for samples that can be transferred after cutting. Embodiments of the present disclosure can be used in imaging application where thin, optically clear membranes are desired.

Current membrane materials for use in laser capture microdissection include polyethylene terephthalate and polyethylene naphthalate (PEN) thin films. These membranes are extremely porous and therefore the optical properties of the membranes are very poor.

It is difficult to image cells on these membranes. The PEN material is designed to be efficient because it absorbs energy in the range of the laser (i.e., about 355 nm). However, it also absorbs light at about 380 nm which prevents, for example, DAPI staining of the cell membranes. DAPI or 4′,6-diamidino-2-phenylindole is a fluorescent stain that binds strongly to DNA. It is used extensively in fluorescence microscopy. Since DAPI will pass through an intact cell membrane, it may be used to stain both live and fixed cells.

For fluorescence microscopy, DAPI is excited with ultraviolet light. When bound to double-stranded DNA, its absorption maximum is at 358 nm and its emission maximum is at 461 nm. (This emission is fairly broad, and appears blue/cyan.) DAPI will also bind to RNA, though it is not as strongly fluorescent. Its emission shifts to around 500 nm when bound to RNA. These are both significant limitations of the current membranes.

Embodiments of the films present disclosure can be easily fabricated (by spin coating, sputtering, etc.) into thin membranes. The material absorbs light up to 360 nm but is nearly transparent to all light above this wavelength, meaning that it is still efficient for cutting with the laser but does not affect fluorescent staining above 360 nm. In addition the membrane is perfectly optically clear, improving visualization of cells.

One aspect of the disclosure encompasses transfer capture membrane for laser microdissection of a biological tissue sample, wherein the membrane can comprise a polymeric composition having the characteristic of being cut by a laser light at a wavelength less than 360 nm, wherein the capture membrane is substantially optically transparent at a wavelength greater than about 360 nm.

In embodiments of the transfer capture membrane of the disclosure, the polymeric composition can comprise a photoreactive epoxy resin.

In one embodiment of the transfer capture membrane of this aspect of the disclosure, where the polymeric composition can comprise the photoreactive epoxy resin SU-8.

In embodiments of the transfer capture membrane of the disclosure, the membrane can have a thickness between about 1 micron and about 20 microns.

In some embodiments of the transfer capture membrane of the disclosure, the membrane can have a thickness between about 1 micron and about 10 microns.

In some embodiments of the transfer capture membrane of the disclosure, the membrane can have a thickness between about 4 microns and about 10 microns.

Another aspect of the disclosure encompasses methods of isolating a cell or population of cells from a tissue, comprising: overlaying a tissue sample with a transfer capture membrane, where the transfer capture membrane comprises a polymeric composition having the characteristic of being cut by a laser light at a wavelength less than 360 nm, and where the capture membrane is substantially optically transparent at a wavelength greater than about 360 nm, and where a cell or a population of cells adheres to the transfer capture membrane; defining an area of the transfer capture membrane with a laser light, where the laser light cuts through the thickness of the transfer capture membrane and the issue sample; and removing the defined area of the transfer capture membrane, thereby isolating the cell or population of cells from the tissue sample.

In embodiments of the methods of this aspect of the disclosure, the polymeric composition can comprise a photoreactive epoxy resin.

In one embodiment of the methods of this aspect of the disclosure, the polymeric composition can comprise the photoreactive epoxy resin SU-8.

In embodiments of the methods of this aspect of the disclosure, the membrane can have a thickness between about 1 micron and about 20 microns.

In embodiments of the methods of this aspect of the disclosure, the membrane can have a thickness between about 1 micron and about 10 microns.

In embodiments of the methods of this aspect of the disclosure, the membrane can have a thickness between about 4 microns and about 10 microns.

Still another aspect of the disclosure provides kits comprising materials to prepare a transfer capture membrane comprising a polymeric composition having the characteristic of being cut by a laser light at a wavelength less than 360 nm, where the capture membrane is substantially optically transparent at a wavelength greater than about 360 nm, packaging, and instructions for combining the materials to form a transfer capture membrane.

It should be emphasized that the embodiments of the present disclosure, particularly, any “preferred” embodiments, are merely possible examples of the implementations, merely set forth for a clear understanding of the principles of the disclosure. Many variations and modifications may be made to the above-described embodiment(s) of the disclosure without departing substantially from the spirit and principles of the disclosure. All such modifications and variations are intended to be included herein within the scope of this disclosure, and the present disclosure and protected by the following claims.

We claim:

1. A transfer capture membrane comprising a polymeric composition having the characteristic of being cut by a laser light at a wavelength less than 360 nm, wherein the capture membrane is substantially optically transparent at a wavelength greater than about 360 nm.

2. The transfer capture membrane of claim 1, wherein the polymeric composition comprises a photoreactive epoxy resin.

3. The transfer capture membrane of claim 1, wherein the polymeric composition comprises the photoreactive epoxy resin SU-8.

4. The transfer capture membrane of claim 1, wherein the membrane has a thickness between about 1 micron and about 20 microns.

5. The transfer capture membrane of claim 1, wherein the membrane has a thickness between about 1 micron and about 10 microns.

6. The transfer capture membrane of claim 1, wherein the membrane has a thickness between about 4 microns and about 10 microns.

7. A method of isolating a cell or population of cells from a tissue, comprising:
overlaying a tissue sample with a transfer capture membrane, wherein the transfer capture membrane comprises a polymeric composition having the characteristic of being cut by a laser light at a wavelength less than 360 nm, and wherein the capture membrane is substantially optically transparent at a wavelength greater than about 360 nm, and wherein a cell or a population of cells adheres to the transfer capture membrane;
defining an area of the transfer capture membrane with a laser light, wherein the laser light cuts through the thickness of the transfer capture membrane and the issue sample; and
removing the defined area of the transfer capture membrane, thereby isolating the cell or population of cells from the tissue sample.

8. The method of claim 7, wherein the polymeric composition comprises a photoreactive epoxy resin.

9. The method of claim 7, wherein the polymeric composition comprises the photoreactive epoxy resin SU-8.

10. The method of claim 7, wherein the membrane has a thickness between about 1 micron and about 20 microns.

11. The method of claim 7, wherein the membrane has a thickness between about 1 micron and about 10 microns.

12. The method of claim 7, wherein the membrane has a thickness between about 4 microns and about 10 microns.

13. A kit comprising materials to prepare a transfer capture membrane comprising a polymeric composition having the characteristic of being cut by a laser light at a wavelength less than 360 nm, wherein the capture membrane is substantially optically transparent at a wavelength greater than about 360 nm, packaging, and instructions for combining the materials to form a transfer capture membrane.
